SEQUENCE LISTING (13 sequences)

SEQ ID NO: 1 - protein, 45 aa, Artificial Sequence
  cartilage-oligomeric matrix protein (COMP) spacer
DLGPQMLRELQETNAALQDVRELLRQQVREITFLKNTVMECDACG

SEQ ID NO: 2 - protein, 450 aa, Homo sapiens
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 3 - protein, 194 aa, Artificial Sequence
  dominant negative C-terminal Src kinase (dnCSK), CSK_del_kinase
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKE

SEQ ID NO: 4 - protein, 132 aa, Artificial Sequence
  dnCSK, CSK_del_kinase_SH3
MSAIQAAWVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKE

SEQ ID NO: 5 - protein, 450 aa, Artificial Sequence
  dnCSK with SH3 domain deleted, CSK(K222R)
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVRCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 6 - protein, 450 aa, Artificial Sequence
  dnCSK with the mutation T266G, CSKas
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVGEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 7 - protein, 256 aa, Artificial Sequence
  sequence of tyrosine kinase domain of CSK
LKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 8 - protein, 108 aa, Artificial Sequence
  dimerization domain, FKBP12 domain
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 9 - protein, 100 aa, Artificial Sequence
  dimerization domain, wild-type FRB segment of mTOR
MASRILWHEMWHEGLEEASRLYFGERNVKGMFEVLEPLHAMMERGPQTLKETSFNQAYGRDLMEAQEWCRKYMKSGNVKDLTQAWDLYYHVFRRISKLES

SEQ ID NO: 10 - protein, 100 aa, Artificial Sequence
  dimerization domain, FRB with T to L substitution at 2098 which allows binding to AP21967
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLES

SEQ ID NO: 11 - protein, 100 aa, Artificial Sequence
  dimerization domain, FRB segment of mTOR with T to H substitution at 2098 and W to F at residue 2101 of the full mTOR which binds Rapamycin with reduced affinity to wild type
MASRILWHEMWHEGLEEASRLYFGERNVKGMFEVLEPLHAMMERGPQTLKETSFNQAYGRDLMEAQEWCRKYMKSGNVKDLHQAFDLYYHVFRRISKLES

SEQ ID NO: 12 - protein, 100 aa, Artificial Sequence
  dimerization domain, FRB segment of mTOR with K to P substitution at residue 2095 of the full mTOR which binds Rapamycin with reduced affinity
MASRILWHEMWHEGLEEASRLYFGERNVKGMFEVLEPLHAMMERGPQTLKETSFNQAYGRDLMEAQEWCRKYMKSGNVPDLTQAWDLYYHVFRRISKLES

SEQ ID NO: 13 - protein, 20 aa, Artificial Sequence
  cleavage site, 2A-like sequence
RAEGRGSLLTCGDVEENPGP

(19) United States
(12) Patent Application Publication
(10) Pub. No.: US 2023/0233606 A1
(43) Pub. Date: Jul. 27, 2023

(54) Cell

(71) Applicant: AUTOLUS LIMITED, London (GB)

(72) Inventors: Shaun Cordoba, London (GB); Evangelia Kokalaki, London (GB); Thomas Grothier, London (GB); Shimobi Onuoha, London (GB); Simon Thomas, London (GB); Martin Pulé, London (GB)

(21) Appl. No.: 17/784,408

(22) PCT Filed: Dec. 18, 2020
(86) PCT No.: PCT/GB2020/053277

§ 371 Date: Jun. 10, 2022

(30) Foreign Application Priority Data
Dec. 20, 2019 (GB) 1919017.2

(51) Int. Cl. A61K 35/17; C12N 9/12; C12N 5/0783; C07K 16/28; C07K 14/705; C07K 14/725

(52) U.S. Cl. CPC A61K 35/17; C12N 9/12; C12Y 207/10002; C12N 5/0636; C07K 16/2803; C07K 14/70578; C07K 14/7051; C07K 2317/622; C07K 2319/03; C07K 2319/33

(57) ABSTRACT

The present invention provides cell which co-expresses a chimeric antigen receptor (CAR) and a dominant negative C-terminal Src kinase (dnCSK). The present invention also provides nucleic acid constructs, vectors and methods for making such a cell and the use of such a cell in the treatment of diseases such as cancer by adoptive immunotherapy.

### FIELD OF THE INVENTION

The present invention relates to a cell which expresses a chimeric antigen receptor (CAR). In particular, it relates to a cell which co-expresses a CAR and a dominant negative C-terminal Src kinase (dnCSK).

### BACKGROUND TO THE INVENTION

Chimeric Antigen Receptors (CARs)

Chimeric antigen receptors are proteins which, in their usual format, graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals.

The most common form of these molecules use single-chain variable fragments (scFv) derived from monoclonal antibodies to recognize a target antigen. The scFv is fused via a spacer and a transmembrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

Although CAR-T cell-mediated treatment have shown success in the clinic towards abundant target antigens such as CD19 or GD2, chimeric antigen receptors have been reported to fail to signal in response to very low-density antigens.

For example, a CAR-T study targeting anaplastic lymphoma kinase (ALK), showed that the CAR-T cells had limited anti-tumor efficacy in two xenograft models of human neuroblastoma. It was shown that cytokine production was highly dependent upon ALK target density and that target density of ALK on neuroblastoma cell lines was insufficient for maximal activation of CAR T cells (Walker et al. (2017) Mol. Ther. 25, 2189-2201).

Another study involved the use of anti-CD22 CAR-T cell in the treatment of relapsed and/or refractory pre-B cell acute lymphoblastic leukemia (B-ALL), although dose-dependent antileukemic activity was observed, some relapses were observed. Relapses were associated with diminished CD22 site density that were thought to permitted CD22+ cell escape from killing by CD22-CAR T cells (Fry et al. (2017) Nat. Med. doi:10.1038/nm.4441).

There is a hierarchy of CAR T-cell activation from killing, to cytokine release to proliferation. A CAR T-cell may kill a target cell with low density antigen but fail to fully activate.

There is therefore a need for alternative CAR T-cell approaches, capable of killing target cells expressing a low density of target antigen.

### DESCRIPTION OF THE FIGURES

FIG. 1: Schematic diagram illustrating LCK, a Src Family Kinase (SFK)

FIG. 2: Schematic diagram illustrating CSK and the dnCSK constructs tested in the Examples.

A—Wild-type CSK having a SH3 domain, an SH2 domain and a protein tyrosine kinase domain.

B—dnCSK lacking a kinase domain

C—dnCSK lacking a kinase domain and an SH3 domain

6—dnCSK having a mutation K222R

FIG. 3: Cytotoxicity assay using target cells expressing high levels of CD22 Results of a killing assay for T-cells expressing an anti-CD22 CAR against target cells with a high target antigen density. T cells were either left untransduced (NT), transduced with an anti-CD22 CAR (LT22), or transduced with a bicistronic construct expressing the CAR together with i) wild-type CSK (LT22+wtCSK); ii) dominant negative CSK lacking the kinase domain (LT22+dCSK (del_kinase)); iii) dominant negative CSK lacking the kinase domain and SH3 domains (LT22+dCSK (del_kinase_SH3); or iv) dominant negative CSK comprising a mutation at amino acid position 222 (LT22+dnCSK (K222R)). CAR expressing cells were co-cultured target cells at a 1:2 or a 1:4 ratio. After 72 hours, killing of target cells was assayed by FACS and the target cell count normalised to counts obtained with untransfected T-cells (NT).

FIG. 4: Cytotoxicity assay using target cells expressing low levels of CD22 Results of a killing assay for T-cells expressing an anti-CD22 CAR against target cells with a low target antigen density. T cells were either left untransduced (NT), transduced with an anti-CD22 CAR (LT22), or transduced with a bicistronic construct expressing the CAR together with i) wild-type CSK (LT22+wtCSK); ii) dominant negative CSK lacking the kinase domain (LT22+dCSK (del_kinase)); iii) dominant negative CSK lacking the kinase domain and SH3 domains (LT22+dCSK (del_kinase_SH3); or iv) dominant negative CSK comprising a mutation at amino acid position 222 (LT22+dnCSK (K222R)). CAR expressing cells were co-cultured target cells at a 1:2 or a 1:4 ratio. After 72 hours, killing of target cells was assayed by FACS and the target cell count normalised to counts obtained with untransfected T-cells (NT).

FIG. 5: IL-2 production by T-cells expressing an CD22 CAR and with or without dnCSK following co-culture with target cells.

CAR expressing cells were co-cultured with high or low expressing CD22 target cells at a 1:4 ratio. Non-transduced (NT) target cells were used as a negative control. T cells were either left untransduced (NT), transduced with an anti-CD22 CAR (LT22), or transduced with a bicistronic construct expressing the CAR together with i) wild-type CSK (LT22+wtCSK); ii) dominant negative CSK lacking the kinase domain (LT22+dCSK (del_kinase)); iii) dominant negative CSK lacking the kinase domain and SH3 domains (LT22+dCSK (del_kinase_SH3); or iv) dominant negative CSK comprising a mutation at amino acid position 222 (LT22+dnCSK (K222R)). After 72 hours IL-2 production was assayed by ELISA.

FIG. 6: IFNγ production by T-cells expressing an CD22 CAR and with or without dnCSK following co-culture with target cells.

CAR expressing cells were co-cultured with high or low expressing CD22 target cells at a 1:4 ratio. Non-transduced (NT) target cells were used as a negative control. T cells were either left untransduced (NT), transduced with an anti-CD22 CAR (LT22), or transduced with a bicistronic construct expressing the CAR together with i) wild-type CSK (LT22+wtCSK); ii) dominant negative CSK lacking the kinase domain (LT22+dCSK (del_kinase)); iii) dominant negative CSK lacking the kinase domain and SH3 domains (LT22+dCSK (del_kinase_SH3); or iv) dominant negative CSK comprising a mutation at amino acid position 222 (LT22+dnCSK (K222R)). After 72 hours IFNγ production was assayed by ELISA.

FIG. 7: Cytotoxicity of LT22 CAR co-expressed with either wtCSK or CSK^{AS}

Analysis by flow cytometry was carried out at 72 h post co-culture set up. Each condition was tested with a minimum of 6 donors (n=6), with median indicated. All data was normalised to NT T cells. T cells were co-cultured with SupT1 NT (left), SupT1 CD22 High (middle) or SupT1 CD22 Low (right) target cells at an E:T ratio of 1:4. Statistics were run using a paired t-test. *P<0.05, **P<0.01, ***P<0.001, ****P<0.0001.

FIG. 8: IL-2 release by LT22 CAR co-expressed with either wtCSK or CSK^{AS}

Supernatant taken at 72 h post co-culture set up was analysed by ELISA for the presence of IL-2. Each condition was tested with a minimum of 6 donors (n=6), with median indicated. T cells were co-cultured with SupT1 NT (left), SupT1 CD22 High (middle) or SupT1 CD22 Low (right) target cells at an E:T ratio of 1:4.

FIG. 9: IFN-γ release by LT22 CAR co-expressed with either wtCSK or CSK^{AS}

Supernatant taken at 72 h post co-culture set up was analysed by ELISA for the presence of IFN-γ. Each condition was tested with a minimum of 6 donors (n=6), with median indicated. T cells were co-cultured with SupT1 NT (left), SupT1 CD22 High (middle) or SupT1 CD22 Low (right) target cells at an E:T ratio of 1:4.

FIG. 10: Cytotoxicity of LT22 CAR co-expressing CSK^{AS} in response to increasing concentrations of the CSK^{AS} inhibitor, 3-IB-PP1

Analysis by flow cytometry was carried out at 72 h post co-culture set up. Each condition was tested with a minimum of 3 donors (n=3), with median indicated. All data was normalised to NT T cells. T cells were co-cultured with SupT1 NT (left) or SupT1 CD22 High (right) target cells at an E:T ratio of 1:4.

FIG. 11: IFN-γ release by LT22 CAR co-expressing CSK^{AS} in response to increasing concentrations of the CSK^{AS} inhibitor, 3-IB-PP1

Analysis by flow cytometry was carried out at 72 h post co-culture set up. Each condition was tested with a minimum of 3 donors (n=3), with median indicated. All data was normalised to NT T cells. T cells were co-cultured with SupT1 NT (left) or SupT1 CD22 High (right) target cells at an E:T ratio of 1:4.

FIG. 12: Schematic diagram illustrating inducible CSK dampener structures and mechanism of action.

a) Four different inducible CSK dampener modules. The four constructs all express the kinase domain of CSK fused to FRB and the SH3 and SH2 domains fused to FKBP12. The orientation of the proteins in these fusions differ between the four constructs.

b) The mechanism of action of the inducible CSK dampener. FRB and FKBP12 are proteins which bind to rapamycin at two distinct epitopes with high affinity. In this system the different domains of CSK are separated: the kinase domain is tethered to FRB and the SH3 and SH2 domains fused to FKBP12. Left: Lck is phosphorylated at Y394 in an active, open conformation, facilitating CAR signalling. The SH3-SH2-FKBP12 component is localised to the membrane via the SH2 domain binding to phosphorylated PAG (acts as dominant-negative). The kinase-FRB component lacks the SH2 domain and thus cannot localise to the membrane via PAG and therefore remains in the cytosol. Right: Upon administration of rapamycin, both FRB and FKBP bind to rapamycin, leading to the recruitment of kinase-FRB recruitment to the membrane. Subsequently, the kinase domain phosphorylates Lck at Y505, inactivating it and inhibiting downstream signalling.

### SUMMARY OF ASPECTS OF THE INVENTION

The present inventors have found that the function of CAR-expressing cells, such as CAR-T cells, can be enhanced by co-expression of a dominant negative C-terminal Src kinase (dnCSK).

Thus, in a first aspect, the present invention provides a cell which co-expresses:

i) a chimeric antigen receptor (CAR); and

(ii) a dominant negative C-terminal Src kinase (dnCSK).

The dnCSK may, for example, be:

i) a truncated CSK which is recruited to the cell membrane but lacks a functional kinase domain;

ii) a mutated CSK which lacks the capacity to phosphorylate Y505 of Lck; or

iii) a mutated CSK whose catalytic activity is inhibited by an agent.

The dnCSK may be a truncated CSK which retains the capacity to bind PAG, Lime and/or Dok1/2 but which lacks a functional kinase domain.

For example, the dnCSK may comprise a CSK SH2 domain but lack a functional kinase domain.

The dnCSK may comprise a CSK SH2 domain and a first dimerisation domain. In connection with this embodiment, the cell may co-express a dampening component comprising a CSK kinase domain and a second dimerization domain which, in the presence of a chemical inducer of dimerization (CID), binds the first dimerisation domain of the dnCSK. In connection with this embodiment, one dimerization domain may comprise an FK506-binding protein (FKBP) and the other dimerization domain may comprise an FRB domain of mTOR and the CID may be rapamycin or a rapamycin analogue.

A mutant dnCSK may lack the capacity to phosphorylate Y505 of Lck. It may have a substitution or deletion at amino acid position K222 with reference to the amino acid numbering of SEQ ID No. 2.

The dnCSK may be a mutated CSK whose catalytic activity is inhibited by an agent. The agent may, for example, be 3-iodo-benzyl-PP1.

The CAR may bind a low density target antigen, such as a target antigen expressed at an average density of fewer than 1500 copies per target cell.

The target antigen may be selected from: ROR1, Tyrp-1, TACl, ALK and BCMA.

In a second aspect, the present invention provides a nucleic acid construct, which comprises:
a first nucleic acid sequence encoding a chimeric antigen receptor; and
a second nucleic acid sequence encoding a dominant negative C-terminal Src kinase (dnCSK) as defined in any preceding claim.

The nucleic acid construct may comprise:
a first nucleic acid sequence encoding a chimeric antigen receptor; and
a second nucleic acid sequence encoding a dominant negative C-terminal Src kinase (dnCSK) which comprise a CSK SH2 domain and a first dimerisation domain; and
a third nucleic acid sequence encoding a dampening component comprising a CSK kinase domain and a second dimerization domain which, in the presence of a chemical inducer of dimerization (CID), binds the first dimerisation domain of the dnCSK.

In a third aspect, the present invention provides vector which comprises a nucleic acid construct according to the second aspect of the invention.

The vector may be a retroviral or lentiviral vector.

In a fourth aspect, the present invention provides a pharmaceutical composition comprising a plurality of cells according to the first aspect of the invention.

In a fifth aspect, the present invention provides a pharmaceutical composition according to the fourth aspect of the invention for use in treating a disease.

In a sixth aspect, the present invention provides a method for treating a disease, which comprises the step of administering a pharmaceutical composition according to fourth aspect of the invention to a subject.

The method may comprise the following steps:
(i) isolation of a cell containing sample from a subject;
(ii) transduction or transfection of the cells with a nucleic acid construct according to second aspect of the invention or a vector according to the third aspect of the invention; and
(iii) administering the cells from (ii) to the subject.

In a seventh aspect, the present invention provides the use of a pharmaceutical composition according to the fourth aspect of the invention in the manufacture of a medicament for the treatment of a disease.

The disease may be cancer.

In an eighth aspect, the present invention provides a method for making a cell according to the first aspect of the invention, which comprises the step of introducing:

a nucleic acid construct according to the second aspect of the invention or a vector according to the third aspect of the invention, into the cell ex vivo.

The cell may be from a sample isolated from a subject.

In a ninth aspect, the present invention provides a method for enhancing the target-antigen sensitivity of a CAR-expressing cell according aspect of the invention in a subject, wherein the cell expresses a mutated CSK whose catalytic activity is inhibited by an agent, which comprises the step of administering the agent to the subject.

In a tenth aspect the present invention provides a method for dampening CAR-mediated activation of a cell in a subject, wherein the cell co-expresses:
(i) a dnCSK comprising a CSK SH2 domain and a first dimerisation domain; and
(ii) a dampening component comprising a CSK kinase domain and a second dimerization domain which, in the presence of a chemical inducer of dimerization (CID), binds the first dimerisation domain of the dnCSK,
which method comprises the step of step of administering the CID to the subject.

### FURTHER ASPECTS

Further aspects of the invention are provided in the following numbered paragraphs:

1. A cell which expresses a chimeric antigen receptor (CAR) and comprises a heterologous nucleic acid sequence encoding C-terminal Src kinase (CSK).

2. A cell according to paragraph 1, wherein expression of the CSK-encoding nucleic acid sequence is inducible.

3. A cell according to paragraph 1 or 2, wherein activity of CSK encoded by the heterologous nucleic acid sequence is inhibited by the presence of an agent.

4. A cell according to paragraph 3, wherein the CSK encoded by the heterologous nucleic acid sequence is a mutated CSK whose catalytic activity is inhibited by 3-iodo-benzyl-PP1.

5. A cell according to any preceding paragraph wherein the CAR binds a target antigen which is expressed at a high level on cancer cells and a low level on one or more normal tissues.

6. A cell according to paragraph 5, wherein the target antigen is selected from: CEA, MUC1, MUC16, EpCAM and ROR1.

7 A nucleic acid construct, which comprises:
a first nucleic acid sequence encoding a chimeric antigen receptor; and
a second nucleic acid sequence encoding C-terminal Src kinase (CSK).

8. A nucleic acid construct according to paragraph 7, wherein the second nucleic acid sequence encodes a mutated CSK whose catalytic activity is inhibited by an agent.

9. A nucleic acid construct according to paragraph 8, wherein the agent is 3-iodo-benzyl-PP1.

10. A nucleic acid construct according to paragraph 8, wherein the agent is rapamycin.

11. A kit of nucleic acid sequences which comprises:
a first nucleic acid sequence encoding a chimeric antigen receptor; and
a second nucleic acid sequence encoding C-terminal Src kinase (CSK).

12. A vector which comprises a nucleic acid construct according to any of paragraphs 7 to 10.

13. A kit of vectors, which comprises:
a first vector comprising a first nucleic acid sequence encoding a chimeric antigen receptor; and
a second vector comprising a second nucleic acid sequence encoding C-terminal Src kinase (CSK).

14. A pharmaceutical composition comprising a plurality of cells according to any of paragraphs 1 to 9.

15. A pharmaceutical composition according to paragraph 14 for use in treating a disease.

16. A method for treating a disease, which comprises the step of administering a pharmaceutical composition according to paragraph 14 to a subject.

17. A method according to paragraph 16, which comprises the following steps:
(i) isolation of a cell containing sample from a subject;
(ii) transduction or transfection of the cells with a nucleic acid construct according to paragraph any of paragraphs 7 to 10; a kit of nucleic acid sequences according to paragraph 11; a vector according to paragraph 12; or a kit of vectors according to paragraph 13; and
(iii) administering the cells from (ii) to the subject.

18. The use of a pharmaceutical composition according to paragraph 14 in the manufacture of a medicament for the treatment and/or prevention of a disease.

19. A pharmaceutical composition for use according to paragraph 15, a method according to paragraph 16 or 17, or a use according to paragraph 18, wherein the disease is cancer.

20. A method for making a cell according to any of paragraphs 1 to 9, which comprises the step of introducing: a nucleic acid construct according to paragraph any of paragraphs 7 to 10; a kit of nucleic acid sequences according to paragraph 11; a vector according to paragraph 12; or a kit of vectors according to paragraph 13, into the cell ex vivo.

21. A method according to paragraph 20, wherein the cell is from a sample isolated from a subject.

22. A method for dampening CAR-mediated signalling of a cell according to paragraph 1 in a subject, wherein the cell expresses a mutated CSK whose catalytic activity is inhibited by an agent, which comprises the step of ceasing to administer the agent to the subject.

Information provided in the detailed description section below also applies to aspects mentioned in the above paragraphs.

### DETAILED DESCRIPTION

Chimeric Antigen Receptors

The present invention relates to a cell which expresses a chimeric antigen receptor.

A classical chimeric antigen receptor (CAR) is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

When the CAR binds the target-antigen, this results in the transmission of an activating signal to the T-cell it is expressed on. Thus the CAR directs the specificity and cytotoxicity of the T cell towards tumour cells expressing the targeted antigen.

CARs typically therefore comprise: (i) an antigen-binding domain; (ii) a spacer; (iii) a transmembrane domain; and (iii) an intracellular domain which comprises or associates with a signalling domain.

A CAR may have the general structure:

Antigen binding domain—spacer domain—transmembrane domain—intracellular signaling domain (endodomain).

Antigen Binding Domain

The antigen binding domain is the portion of the CAR which recognizes antigen. In a classical CAR, the antigen-binding domain comprises: a single-chain variable fragment (scFv) derived from a monoclonal. CARs have also been produced with domain antibody (dAb), VHH or Fab-based antigen binding domains.

Alternatively a CAR may comprise a ligand for the target antigen. For example, B-cell maturation antigen (BCMA)-binding CARs have been described which have an antigen binding domain based on the ligand a proliferation inducing ligand (APRIL).

Spacer

Classical CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

A variety of sequences are commonly used as spacers for CAR, for example, an IgG1 Fc region, an IgG1 hinge, or a human CD8 stalk.

WO2016/151315 describes spacers which form coiled-coil domains and form multimeric CARs. For example, it describes a spacer based on the cartilage-oligomeric matrix protein (COMP) which forms pentamers. A COMP spacer may comprise the sequence shown as SEQ ID No. 1 or a truncated version thereof which retains the capacity to form coiled-coils and therefore multimers.

(COMP spacer)
SEQ ID No. 1
DLGPQMLRELQETNAALQDVRELLRQQVREITFLKN
TVMECDACG

Transmembrane Domain

The transmembrane domain is the portion of the CAR which spans the membrane. The transmembrane domain may be any protein structure which is thermodynamically stable in a membrane. This is typically an alpha helix comprising of several hydrophobic residues. The transmembrane domain of any transmembrane protein can be used to supply the transmembrane portion of the CAR. The presence and span of a transmembrane domain of a protein can be determined by those skilled in the art using the TMHMM algorithm (http://www.cbs.dtu.dk/services/TMHMM-2.0/). Alternatively, an artificially designed TM domain may be used.

Endodomain

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signalling may be needed. Co-stimulatory signals promote T-cell proliferation and survival. There are two main types of co-stimulatory signals: those that belong the Ig family (CD28, ICOS) and the TNF family (OX40, 41BB, CD27, GITR etc). For example, chimeric CD28 and OX40 can be used with CD3-Zeta to transmit a proliferative/survival signal, or all three can be used together.

The endodomain may comprise:

(i) an ITAM-containing endodomain, such as the endodomain from CD3 zeta; and/or

(ii) a co-stimulatory domain, such as the endodomain from CD28 or ICOS; and/or

(iii) a domain which transmits a survival signal, for example a TNF receptor family endodomain such as OX-40, 4-1BB, CD27 or GITR.

A number of systems have been described in which the antigen recognition portion is on a separate molecule from the signal transmission portion, such as those described in WO015/150771; WO2016/124930 and WO2016/030691. The CAR of the present invention may therefore comprise an antigen-binding component comprising an antigen-binding domain and a transmembrane domain; which is capable of interacting with a separate intracellular signalling component comprising a signalling domain.

The vector of the invention may express a CAR signalling system comprising such an antigen-binding component and intracellular signalling component.

The CAR may comprise a signal peptide so that when it is expressed inside a cell, the nascent protein is directed to the endoplasmic reticulum and subsequently to the cell surface, where it is expressed. The signal peptide may be at the amino terminus of the molecule.

Target Antigen

A ‘target antigen’ is an entity which is specifically recognised and bound by the antigen-binding domain of a CAR.

The target antigen may be an antigen present on a cancer cell, for example a tumour-associated antigen.

Various tumour associated antigens (TAA) are known, as shown in the following Table 1. The CAR may be capable of binding such a TAA.

TABLE 1

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

The target antigen for the CAR may be expressed at relatively low density on the target cell.

The cells of the present invention may be capable of killing target cells, such as cancer cells, which express a low density of the CAR target antigen. Examples of tumour associated antigens which are known to be expressed at low densities in certain cancers include, but are not limited to, ROR1 in CLL, Typr-1 in melanoma, BCMA, and TACI in myeloma and ALK in Neuroblastoma.

The mean copy number of the target antigen for the CAR may be fewer than about 10,000; 5,000; 3,000; 2,000; 1,000; or 500 copies per target cell.

The copy number of an antigen on a cell, such as a cancer cell may be measured using standard techniques, such as using PE Quantibrite beads.

The target antigen for the CAR may be expressed by the target cell at an average copy number of 1500 copies per cell or fewer, or 1000 copies per cell or fewer.

The target antigen may, for example, be BCMA, ROR1, Tyrp-1, TACI or ALK

CSK

The cells of the present invention express a dominant negative C-terminal Src kinase (dnCSK).

C-terminal Src kinase, also known as Tyrosine-protein kinase, is an enzyme which phosphorylates tyrosine residues located in the C-terminal end of Src-family kinases (SFKs) including SRC, HCK, FYN, LCK, LYN and YES1, thus suppressing their activity.

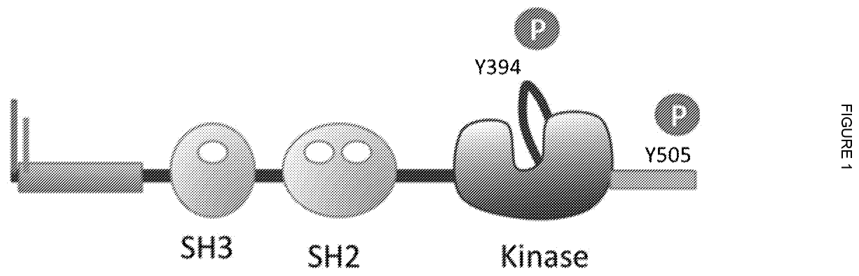

Src Family Kinases (SFKs), such as Lck, are made up of a N-terminal myristoyl group, that permits membrane localisation, attached to an SH4 domain, an SH3 domain, an SH2 domain and a protein tyrosine kinase domain (SH1 domain). Lck is illustrated schematically in FIG. 1.

There is a conserved tyrosine residue in the activation loop and one in the C-terminal tail, phosphorylation of the activation loop tyrosine by trans-autophosphorylation increases SFK activity, whereas phosphorylation of the C-terminal tyrosine by C-terminal Src kinase (CSK) inhibits SFK activity

Csk phosphorylates the negative regulatory C-terminal tyrosine residue Y505 of Lck to maintain Lck in an inactive state. In resting T cells, Csk is targeted to lipid rafts through engagement of its SH2 domain with phosphotyrosine residue pY317 of PAG. PAG is expressed as a tyrosine phosphorylated protein in nonstimulated T-cells. This interaction of Csk and PAG allows activation of Csk and inhibition of Lck.

Upon TCR activation, CD45 is excluded from membrane microdomains and dephosphorylates PAG, leading to Csk detaching from the plasma membrane.

The amino acid sequence of human CSK is available from Uniprot Accession No 41240 and is shown below as SEQ ID No. 2. In this sequence, residues 9-70 correspond to the SH3 domain, residues 82-171 correspond to the SH2 domain; and residues 195-449 correspond to the protein kinase domain.

(wtCSK)
SEQ ID No. 2
MSAIQAAWPSGTECIAKYNFHGTAEQDLPFCKGDV
LTIVAVTKDPNWYKAKNKVGREGIIPANYVQKREG
VKAGTKLSLMPWFHGKITREQAERLLYPPETGLFL
VRESTNYPGDYTLCVSCDGKVEHYRIMYHASKLSI
DEEVYFENLMQLVEHYTSDADGLCTRLIKPKVMEG
TVAAQDEFYRSGWALNMKELKLLQTIGKGEFGDVM
LGDYRGNKVAVKCIKNDATAQAFLAEASVMTQLRH
SNLVQLLGVIVEEKGGLYIVTEYMAKGSLVDYLRS
RGRSVLGGDCLLKFSLDVCEAMEYLEGNNFVHRDL
AARNVLVSEDNVAKVSDFGLTKEASSTQDTGKLPV
KWTAPEALREKKFSTKSDVWSFGILLWEIYSFGRV
PYPRIPLKDVVPRVEKGYKMDAPDGCPPAVYEVMK
NCWHLDAAMRPSFLQLREQLEHIKTHELHL

dnCSK

The cells of the present invention express a dominant negative C-terminal Src kinase (dnCSK).

The dominant negative CSK may lack a functional protein kinase domain. The dnCSK may not comprise a kinase domain or it may comprise a partially or completely inactive kinase domain. The kinase domain may be inactivated by, for example, truncation or mutation of one or more amino acids.

The dnCSK may, for example, be:
i) a truncated CSK which is recruited to the cell membrane but lacks a functional kinase domain;
ii) a mutated CSK which lacks the capacity to phosphorylate Y505 of Lck; or
iii) a mutated CSK whose catalytic activity is inhibited by an agent.

Truncated Kinase Domain

The dnCSK of the present invention may completely lack a kinase domain. For example, the dnCSK may comprise the SH2 domain and optionally the SH3 domain, but be truncated to remove the kinase domain.

Alternatively, the dnCSK of the present invention may comprise a partially truncated kinase domain which comprises part of a phosphatase, for example a portion of the sequence from residues 195-449 of SEQ ID No. 2, provided that the truncated kinase has reduced capacity to phosphorylate the C-terminal tyrosine residue Y505 of Lck compared to wild-type CSK. The truncated kinase may have effectively no residual kinase activity.

The dnCSK may be a truncated CSK which retains the capacity to bind a transmembrane adaptor protein such as PAG, Lime and/or Dok1/2 which recruits wild-type CSK to the cell membrane but lacks a functional kinase domain.

The dnCSK of the present invention may have the sequence shown as SEQ ID No. 3, which corresponds to the wild-type CSK sequence (SEQ ID No. 1) minus the kinase domain.

(CSK_del_kinase)
SEQ ID No. 3
MSAIQAAWPSGTECIAKYNFHGTAEQDLPFCKGDV
LTIVAVTKDPNWYKAKNKVGREGIIPANYVQKREG
VKAGTKLSLMPWFHGKITREQAERLLYPPETGLFL
VRESTNYPGDYTLCVSCDGKVEHYRIMYHASKLSI
DEEVYFENLMQLVEHYTSDADGLCTRLIKPKVMEG
TVAAQDEFYRSGWALNMKE

Alternatively the dnCSK of the present invention may have the sequence shown as SEQ ID No. 4, which corresponds to the wild-type CSK sequence (SEQ ID No. 1) minus the kinase and SH3 domains.

(CSK_del_kinase_SH3)
SEQ ID No. 4
MSAIQAAVKAGTKLSLMPWFHGKITREQAERLLYP
PETGLFLVRESTNYPGDYTLCVSCDGKVEHYRIMY
HASKLSIDEEVYFENLMQLVEHYTSDADGLCTRLI
KPKVMEGTVAAQDEFYRSGWALNMKE

Inactivated Kinase Domain

The dnCSK of the present invention may comprise a kinase domain which is inactivated so that it has reduced or no capacity to phosphorylate proteins such as Lck.

The kinase domain may, for example, comprise one or more amino acid mutations such that it has reduced kinase activity compared to the wild-type sequence.

The mutation may, for example, be an addition, deletion or substitution.

The mutation may comprise the deletion or substitution of one or more lysine residues.

The variant kinase sequence may have a mutation to lysine at position 222 with reference to the sequence shown as SEQ ID No. 2.

The dnCSK of the invention may have the sequence shown as SEQ ID No 4, which corresponds to the full length CSK sequence with a K222R substitution. This mutation is shown in bold and underlined in SEQ ID No. 5. Alternatively, the dnCSK of the invention may have a sequence equivalent to SEQ ID No. 5 in which the SH3 domain has been deleted.

(CSK(K222R))
SEQ ID No 5
MSAIQAAWPSGTECIAKYNFHGTAEQDLPFCKGDV
LTIVAVTKDPNWYKAKNKVGREGIIPANYVQKREG
VKAGTKLSLMPWFHGKITREQAERLLYPPETGLFL
VRESTNYPGDYTLCVSCDGKVEHYRIMYHASKLSI
DEEVYFENLMQLVEHYTSDADGLCTRLIKPKVMEG
TVAAQDEFYRSGWALNMKELKLLQTIGKGEFGDVM
LGDYRGNKVAVRCIKNDATAQAFLAEASVMTQLRH
SNLVQLLGVIVEEKGGLYIVTEYMAKGSLVDYLRS
RGRSVLGGDCLLKFSLDVCEAMEYLEGNNFVHRDL
AARNVLVSEDNVAKVSDFGLTKEASSTQDTGKLPV
KWTAPEALREKKFSTKSDVWSFGILLWEIYSFGRV
PYPRIPLKDVVPRVEKGYKMDAPDGCPPAVYEVMK
NCWHLDAAMRPSFLQLREQLEHIKTHELHL

Conditionally Inactive Kinase Domain

The dnCSK may comprise a mutated CSK whose catalytic activity is inhibited by an agent. For example, the dnCSK may have the sequence shown as SEQ ID No. 6, which comprises the mutation T266G compared to the wildtype sequence shown as SEQ ID No. 2 and is known as “CSKas”. The substitution is in bold and underlined in SEQ ID No. 6. Alternatively, the dnCSK of the invention may have a sequence equivalent to SEQ ID No. 6 in which the SH3 domain has been deleted.

(CSKas)
SEQ ID No.6
MSAIQAAWPSGTECIAKYNFHGTAEQDLPFCKGDV
LTIVAVTKDPNWYKAKNKVGREGIIPANYVQKREG
VKAGTKLSLMPWFHGKITREQAERLLYPPETGLFL
VRESTNYPGDYTLCVSCDGKVEHYRIMYHASKLSI
DEEVYFENLMQLVEHYTSDADGLCTRLIKPKVMEG
TVAAQDEFYRSGWALNMKELKLLQTIGKGEFGDVM
LGDYRGNKVAVKCIKNDATAQAFLAEASVMTQLRH
SNLVQLLGVIVEEKGGLYIVGEYMAKGSLVDYLRS
RGRSVLGGDCLLKFSLDVCEAMEYLEGNNFVHRDL
AARNVLVSEDNVAKVSDFGLTKEASSTQDTGKLPV
KWTAPEALREKKFSTKSDVWSFGILLWEIYSFGRV
PYPRIPLKDVVPRVEKGYKMDAPDGCPPAVYEVMK
NCWHLDAAMRPSFLQLREQLEHIKTHELHL

The catalytic activity of CSKas is inhibited by 3-iodo-benzyl-PP1. In the presence of this molecule, therefore CSKas acts as a dominant negative version of CSK, competing with the wild-type enzyme for binding to membrane proteins such as PAG, Lime and/or Dok1/2 which recruit wild-type CSK to the cell membrane.

Inducible CSK Dampeners

The cell of the invention may co-express:
(i) a dnCSK comprising a CSK SH2 domain (but lacking a kinase domain) and a first dimerisation domain; and
(ii) a dampening component comprising a CSK kinase domain and a second dimerization domain which, in the presence of a chemical inducer of dimerization (CID), binds the first dimerisation domain of the dnCSK,

Various configurations for the dnCSK and dampening component are illustrated schematically in FIG. 12a and the mechanism of action is illustrated schematically in FIG. 12b. In the presence of the CID (which is shown as rapamycin in FIG. 12b), the kinase domain is brought to the cell membrane where it phosphorylates Lck, inhibiting CAR-mediated T-cell signalling.

The dampening component may inhibit CAR-mediated cell signalling completely, effectively “turning off” CAR mediated cell activation. Alternatively, the dampening component may cause partial inhibition, effectively “turning down” CAR-mediated cell signalling.

The presence of the dampening component may result in signalling through the CAR which is 2, 5, 10, 50, 100, 1,000 or 10,000-fold lower than the signalling which occurs in the absence of the dampening component.

CAR mediated signalling may be determined by a variety of methods known in the art. Such methods include assaying signal transduction, for example assaying levels of specific protein tyrosine kinases (PTKs), breakdown of phosphatidylinositol 4,5-biphosphate (PIP2), activation of protein kinase C (PKC) and elevation of intracellular calcium ion concentration. Functional readouts, such as clonal expansion of T cells, upregulation of activation markers on the cell surface, differentiation into effector cells and induction of cytotoxicity or cytokine (e.g. IL-2) secretion may also be utilised.

The dampening component may comprise any kinase domain capable of phosphorylating Lck. The dampening component may comprise the CSK kinase domain having the sequence shown as SEQ ID No. 7.

sequence of tyrosine kinase domain of CSK
SEQ ID No: 7
LKLLQTIGKGEFGDVMLGDYRGNKVAVKCIKNDAT
AQAFLAEASVMTQLRHSNLVQLLGVIVEEKGGLYI
VTEYMAKGSLVDYLRSRGRSVLGGDCLLKFSLDVC
EAMEYLEGNNFVHRDLAARNVLVSEDNVAKVSDFG
LTKEASSTQDTGKLPVKWTAPEALREKKFSTKSDV
WSFGILLWEIYSFGRVPYPRIPLKDVVPRVEKGYK
MDAPDGCPPAVYEVMKNCWHLDAAMRPSFLQLREQ
LEHIKTHELHL

The dnCSK and dampening component dimerise in the presence of an chemical “inducer” of dimerization (CID).

The macrolides rapamycin and FK506 act by inducing the heterodimerization of cellular proteins. Each drug binds with a high affinity to the FKBP12 protein, creating a drug-protein complex that subsequently binds and inactivates mTOR/FRAP and calcineurin, respectively. The FKBP-rapamycin binding (FRB) domain of mTOR has been defined and applied as an isolated 89 amino acid protein moiety that can be fused to a protein of interest. Rapamycin can then induce the approximation of FRB fusions to FKBP12 or proteins fused with FKBP 12.

In the context of the present invention, one of the dimerization domains may comprise FRB or a variant thereof and the other dimerization domain may comprise FKBP12 or a variant thereof.

The dimerization domains may be or comprise one the sequences shown as SEQ ID NO: 8 to SEQ ID NO: 12.

FKBP12 domain
SEQ ID No: 8
MGVQVETISPGDGRTFPKRGQTCVVHYTGMLEDGK
KFDSSRDRNKPFKFMLGKQEVIRGWEEGVAQMSVG
QRAKLTISPDYAYGATGHPGIIPPHATLVFDVELL
KLE
wild-type FRB segment of mTOR
SEQ ID No: 9
MASRILWHEMWHEGLEEASRLYFGERNVKGMFEVL
EPLHAMMERGPQTLKETSFNQAYGRDLMEAQEWCR
KYMKSGNVKDLTQAWDLYYHVFRRISKLES

SEQ ID No: 10—FRB with T to L substitution at 2098 which allows binding to AP21967

MASRILWHEMWHEGLEEASRLYFGERNVKGMFEVL
EPLHAMMERGPQTLKETSFNQAYGRDLMEAQEWCR
KYMKSGNVKDLLQAWDLYYHVFRRISKLES

SEQ ID No 11: —FRB segment of mTOR with T to H substitution at 2098 and to W at F at residue 2101 of the full mTOR which binds Rapamycin with reduced affinity to wild type

MASRILWHEMWHEGLEEASRLYFGERNVKGMFEVL
EPLHAMMERGPQTLKETSFNQAYGRDLMEAQEWCR
KYMKSGNVKDLHQAFDLYYHVFRRISKLES

SEQ ID No 12: —FRB segment of mTOR with K to P substitution at residue 2095 of the full mTOR which binds Rapamycin with reduced affinity

MASRILWHEMWHEGLEEASRLYFGERNVKGMFEVL
EPLHAMMERGPQTLKETSFNQAYGRDLMEAQEWCR
KYMKSGNVPDLTQAWDLYYHVFRRISKLES

Variant sequences may have at least 80%, 85%, 90%, 95%, 98% or 99% sequence identity to SEQ ID No. 8 to 12, provided that the sequences provide an effective dimerization system. That is, provided that the sequences facilitate co-localisation of damening component and the dnCSK at the call membrane.

The “wild-type” FRB domain shown as SEQ ID No. 9 comprises amino acids 2025-2114 of human mTOR. Using the amino acid numbering system of human mTOR, the FRB sequence may comprise an amino acid substitution at one of more of the following positions: 2095, 2098, 2101.

The variant FRB may comprise one of the following amino acids at positions 2095, 2098 and 2101:

2095: K, P, T or A

2098: T, L, H or F

2101: W or F

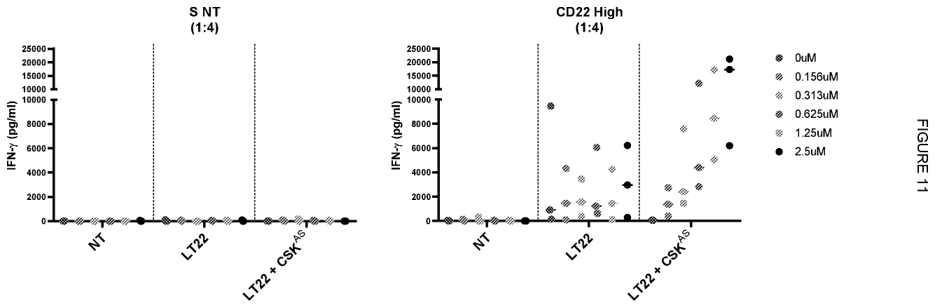

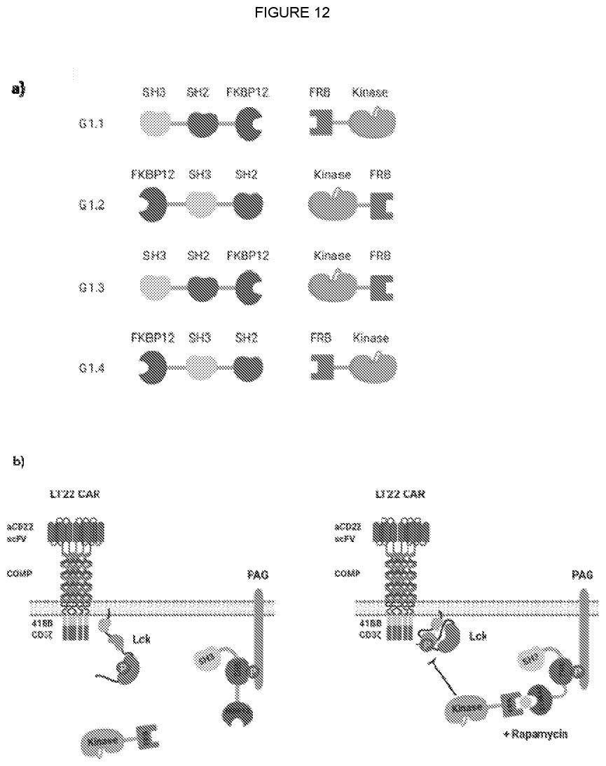

Bayle et al (Chem Bio; 2006; 13; 99-107) describe the following FRB variants, annotated according to the amino acids at positions 2095, 2098 and 2101 (see Table 1): KTW, PLF, KLW, PLW, TLW, ALW, PTF, ATF, TTF, KLF, PLF, TLF, ALF, KTF, KHF, KFF, KLF. These variants are capable of binding rapamycin and rapalogs to varying extents, as shown in Table 1 and FIG. 5A of Bayle et al. The MTC or SDC of the cell of the invention may comprise one of these FRB variants.

In order to prevent rapamycin binding and inactivating endogenous mTOR, the surface of rapamycin which contacts FRB may be modified. Compensatory mutation of the FRB domain to form a burface that accommodates the “bumped” rapamycin restores dimerizing interactions only with the FRB mutant and not to the endogenous mTOR protein.

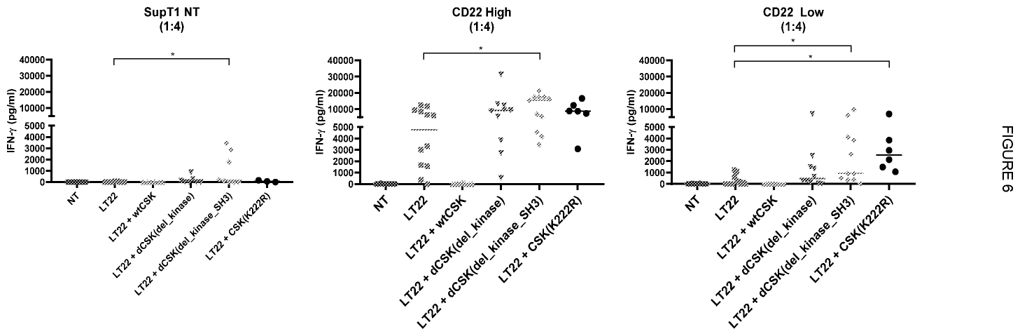

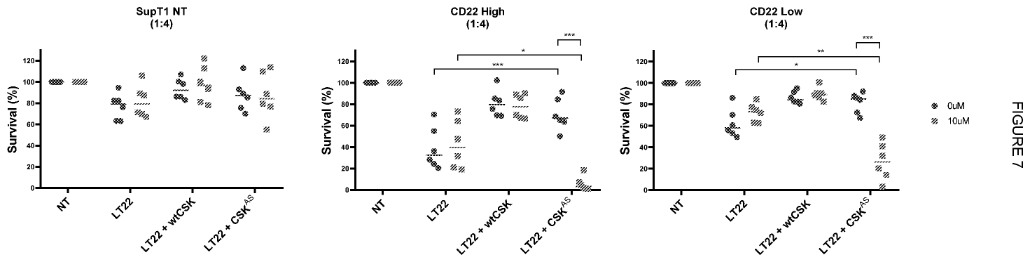

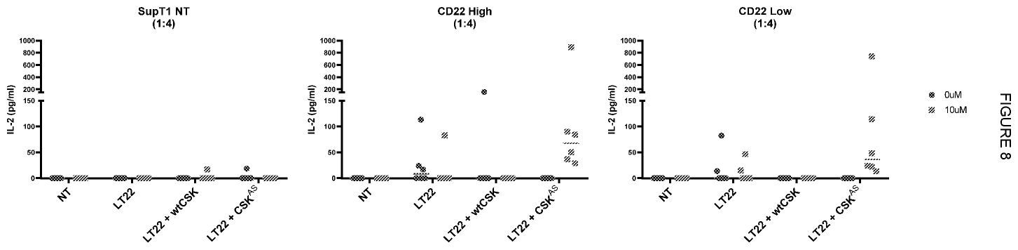

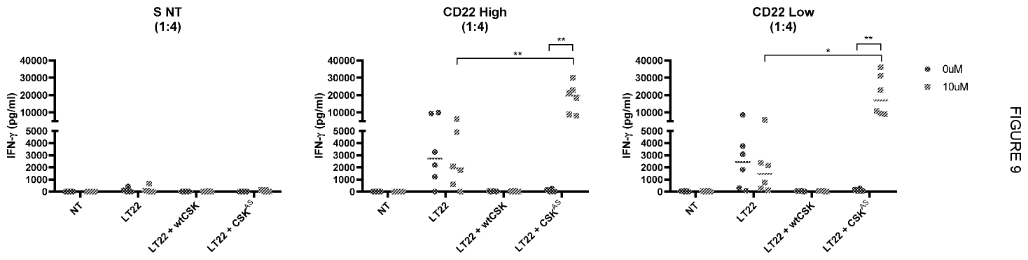

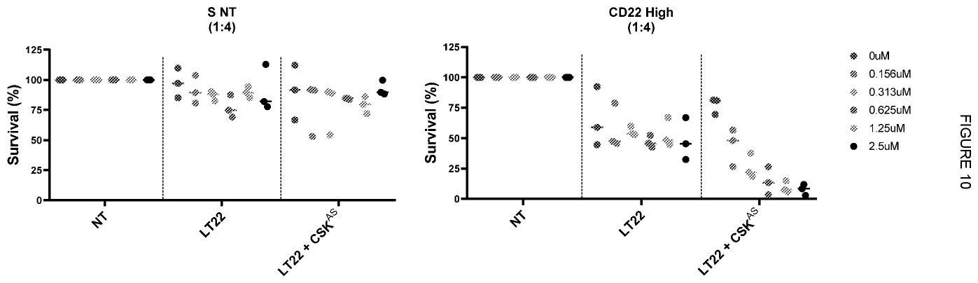

Bayle et al. (as above) describe various rapamycin analogs, or “rapalogs” and their corresponding modified FRB binding domains. For example: C methyllyrlrapamycin (MaRap), C16(5)-Butylsulfonamidorapamycin (C16-BS-Rap) and C16-(S)-7-methylindolerapamycin (AP21976/C16-AiRap), as shown in FIG. 3, in combination with the respective complementary binding domains for each. Other rapamycins/rapalogs include sirolimus and tacrolimus.

Nucleic Acid Sequence

The present invention also provides a nucleic acid sequence encoding a dominant negative CSK.

The nucleic acid sequence may encode the SH2 domain and optionally the SH3 domain of CSK, but not the kinase domain. The nucleic acid sequence may also encode a dimerization domain.

Alternatively, the nucleic acid sequence may encode the SH2 domain and optionally the SH3 domain of CSK, and a mutated kinase domain which is either constitutively inactive, or conditionally inactive in the presence of an agent. The agent may, for example, be 3-iodo-benzyl-PP1.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Nucleic Acid Construct

The present invention also provides a nucleic acid construct encoding a chimeric antigen receptor (CAR) and a dominant negative CSK of the invention.

A nucleic acid construct may have the structure:

dnCSK-coexpr-AgB-spacer-TM-endo, or

AgB-spacer-TM-endo-coexpr-dnCSK

in which:

dnCSK is a nucleic acid sequence encoding the dominant negative CSK;

coexpr is a sequence enabling the coexpression of the dnCSK and CAR as two separate polypeptides;

AgB is a nucleic acid sequence encoding the antigen-binding domain of the CAR;

spacer is a nucleic acid sequence encoding the spacer domain of the CAR;

TM is a nucleic acid sequence encoding the transmembrane domain of the CAR;

endo is a nucleic acid sequence encoding the endodomain of the CAR.

In the structure above, “coexpr” is a nucleic acid sequence enabling co-expression of two polypeptides as separate entities. It may be a sequence encoding a cleavage site, such that the nucleic acid construct produces both polypeptides, joined by a cleavage site(s). The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FM DV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID No. 13

(RAEGRGSLLTCGDVEENPGP).

The present invention also provides a nucleic acid construct comprising:
a first nucleic acid sequence encoding a chimeric antigen receptor; and
a second nucleic acid sequence encoding a dominant negative C-terminal Src kinase (dnCSK) which comprise a CSK SH2 domain and a first dimerisation domain; and
a third nucleic acid sequence encoding a dampening component comprising a CSK kinase domain and a second dimerization domain which, in the presence of a chemical inducer of dimerization (CID), binds the first dimerisation domain of the dnCSK.

Various designs for the dnCSK and dampening component are illustrated schematically in FIG. 12.

A nucleic acid construct may have the structure:

dnCSK-coexpr1-CAR-coexpr2-DC,

dnCSK-coexpr1-DC-coexpr2-CAR,

CAR-coexpr1-dnCSK-coexpr2-DC,

CAR-coexpr1-DC-coexpr2-dnCSK,

DC-coexpr1-dnCSK-coexpr2-CAR, or

DC-coexpr1-CAR-coexpr2-dnCSK

in which:

dnCSK is a nucleic acid sequence encoding the dominant negative CSK;

coexpr1 and coexpr2, which may be the same or different, are a sequences enabling the coexpression of the dnCSK, dampening component and CAR as three separate polypeptides;

DC is a nucleic acid sequence encoding the dampening component.

Vector

The present invention also provides a vector, or kit of vectors, which comprises one or more nucleic acid sequence(s) or nucleic acid construct(s) according to the invention. Such a vector may be used to introduce the nucleic acid sequence(s) into a host cell so that it expresses a CAR according and/or a dominant negative CSK.

A kit of vectors may comprise a first vector which comprises a nucleic acid sequence encoding a CAR and a second vector which comprises a nucleic acid sequence encoding a dnCSK.

The vector may, for example, be a plasmid or a viral vector, such as a retroviral vector or a lentiviral vector, or a transposon based vector or synthetic mRNA.

The vector may be capable of transfecting or transducing a cell, such as a T cell or a NK cell.

Cell

The present invention provides a cell which comprises a chimeric antigen receptor and a dnCSK.

The cell may comprise a nucleic acid sequence, a nucleic acid construct or a vector of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+Treg cells have been described—naturally occurring Treg cells and adaptive Treg cells.

Naturally occurring Treg cells (also known as CD4+CD25+FoxP3+Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

Cells according to the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, cells may be derived from ex vivo differentiation of inducible progenitor cells or embryonic progenitor cells to, for example, T or NK cells. Alternatively, an immortalized T-cell line which retains its lytic function and could act as a therapeutic may be used.

In all these embodiments, chimeric polypeptide-expressing cells are generated by introducing DNA or RNA coding for the chimeric polypeptide by one of many means including transduction with a viral vector, transfection with DNA or RNA.

The cell of the invention may be an ex vivo cell from a subject. The cell may be from a peripheral blood mononuclear cell (PBMC) sample. The cells may be activated and/or expanded prior to being transduced with nucleic acid encoding the molecules providing the chimeric polypeptide according to the first aspect of the invention, for example by treatment with an anti-CD3 monoclonal antibody.

The cell of the invention may be made by:
(i) isolation of a cell-containing sample from a subject or other sources listed above; and
(ii) transduction or transfection of the cells with one or more a nucleic acid sequence(s), nucleic acid construct(s) or vector(s) such that it co-expresses a CAR and a dnCSK.

The cells may then by purified, for example, selected on the basis of expression of the antigen-binding domain of the antigen-binding polypeptide.

Pharmaceutical Composition

The present invention also relates to a pharmaceutical composition containing a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. Herein such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing or transfecting such cells with a nucleic acid sequence or vector provided by the present invention;
(iii) administering the cells from (ii) to a subject.

The cell-containing sample may be isolated from a subject or from other sources, as described above.

The present invention provides a cell of the present invention for use in treating and/or preventing a disease.

The invention also relates to the use of a cell of the present invention in the manufacture of a medicament for the treatment of a disease.

The disease to be treated by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The disease may be Multiple Myeloma (MM), B-cell Acute Lymphoblastic Leukaemia (B-ALL), Chronic Lymphocytic Leukaemia (CLL), Neuroblastoma, T-cell acute Lymphoblastic Leukaema (T-ALL) or diffuse large B-cell lymphoma (DLBCL).

The disease may be a plasma cell disorder such as plasmacytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance or smoldering multiple myeloma.

The cells of the present invention may be capable of killing target cells, such as cancer cells.

The cells and pharmaceutical compositions of present invention may be for use in the treatment and/or prevention of the diseases described above.

The invention will now be further described by way of Examples, which are meant to serve to assist one of ordinary skill in the art in carrying out the invention and are not intended in any way to limit the scope of the invention.

### EXAMPLES

### Example 1—Generation of a Panel of Cells Co-Expressing an Anti-CD22 CAR and Various Alternative Dominant Negative CSKs

A panel of CAR-encoding nucleic acid constructs were generated as follows:

LT22_LH-COM P-TyrpTM-41BBz—expressing the CD22 CAR only

LT22_ LH-COM P-TyrpTM-41BBz-2A-wtCSK—co-expressing the CD22 CAR and wild-type CSK

LT22_LH-COMP-TyrpTM-41BBz-2A-dCSK(del_kinase)—co-expressing the CD22 CAR and dominant negative CSK, lacking a kinase domain

LT22_LH-COMP-TyrpTM-41BBz-2A-dCSK(del_kinase_SH3)—co-expressing the CD22 CAR and dominant negative CSK, lacking a kinase domain and an SH3 domain

LT22_LH-COMP-TyrpTM-41BBz-2A-CSK(K222R)—co-expressing the CD22 CAR and dominant negative CSK, having a K222R mutation.

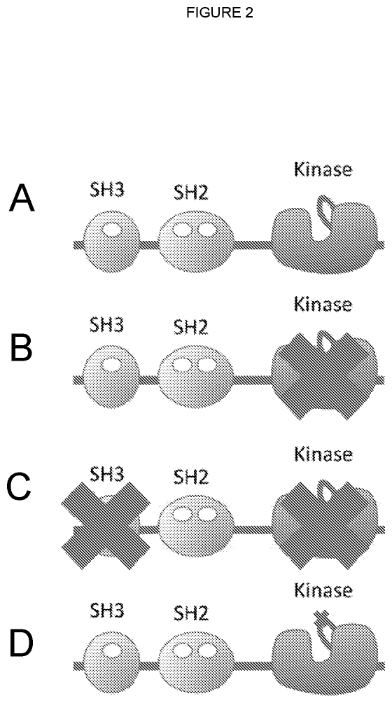

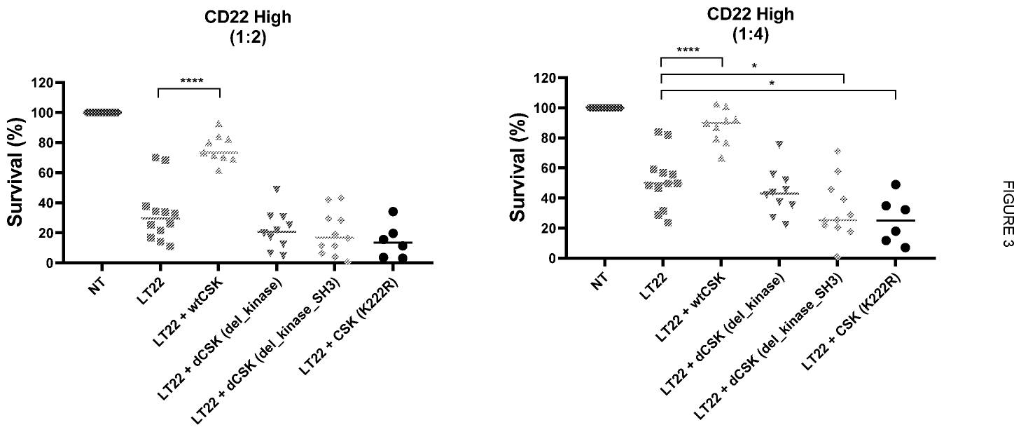

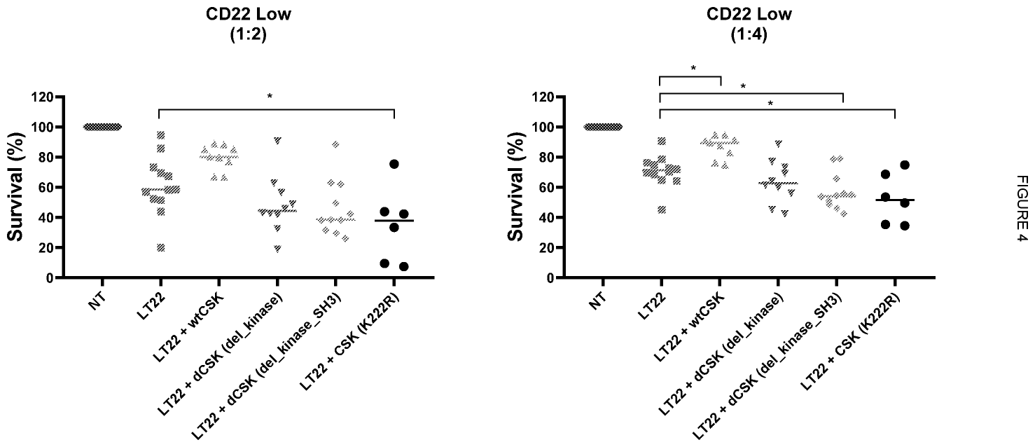

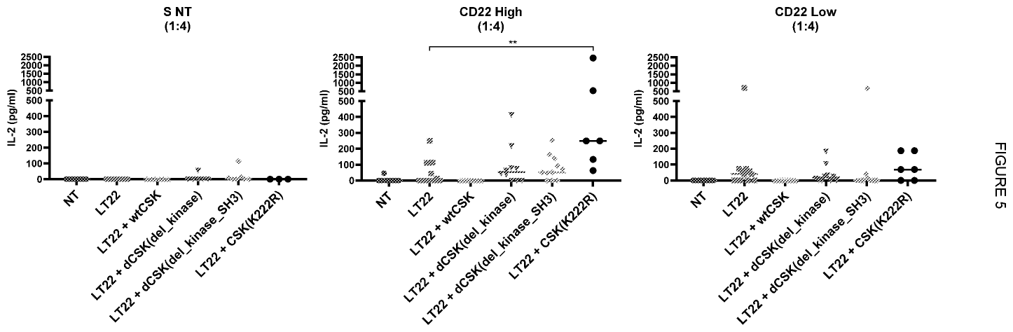

These constructs are illustrated schematically in FIG. 2.

The anti-CD22 CAR had an scFv and antigen-binding domain including the VH and VL sequences from the anti-CD22 mAb LT22; a coiled-coil spacer derived from the protein COMP having the sequence given above as SEQ ID No. 1; a transmembrane domain from the protein Tyrp1; and a second generation compound endodomain, comprising the endodomain of 4-1BB and CD3ζ.

PBMCs were either left untransduced or transduced with one of the constructs.

### Example 2—FACs-Based Killing Assay (FBK)

The capacity of the CAR-T cells to kill target cells expressing CD22 was investigated using a FACS-based killing assay. Target cells which did not express CD22 were used as a negative control. Two different types of CD22-expressing SupT1 target cells were generated, one expressing CD22 at a low density (average number of CD22 molecules per cell: 1968) and one expressing CD22 at a high density (average number of CD22 molecules per cell: 6,309). T-cells were co-cultured with target cells at effector to target ratios of 1:2 and 1:4. Assays were carried out in a 96-well plate in 0.2 ml total volume using 5×10⁴ transduced target cells per well. The co-cultures are set up after being normalised for transduction efficiency. FBK was assayed after 72 h of incubation.

The results are shown in FIGS. 3 (high-density CD22 target cells) and 4 (low-density CD22 target cells). T cells expressing the CAR only showed some level of killing of both the CD22 high and low-expressing target cells, at both at a 1:2 and 1:4 ratio. For both target cell types and at both E:T ratios, target cell killing was considerably dampened by co-expression of wild-type CSK. By contrast, co-expression of any of the three dominant negative CSKs, i.e.: i) dominant negative CSK lacking the kinase domain (LT22+dCSK (del_kinase)); ii) dominant negative CSK lacking the kinase domain and SH3 domains (LT22+dCSK (del_kinase_SH3); or iii) dominant negative CSK comprising a mutation at amino acid position 222 (LT22+dnCSK (K222R)) increased the efficiency of target cell killing.

### Example 3—Cytokine Release

Secretion of IL-2 and Interferon-gamma (IFNγ) by CAR T-cells was measured by collecting supernatant at 72 hr from co-cultures, as described in Example 2, at a 1:4 effector: target ratio using untransduced (NT) target cells; high-expressing CD22 target cells (CD22 High); and low-expressing CD22 target cells (CD22 Low). Production of IL-2 and IFN-G was detected by ELISA.

The results for IL-2 are show in in FIG. 5 and the results for IFNγ are shown in FIG. 6. T cells co-expressing any of the dominant negative CSKs, i.e.: i) dominant negative CSK lacking the kinase domain (LT22+dCSK (del_kinase)); ii) dominant negative CSK lacking the kinase domain and SH3 domains (LT22+dCSK (del_kinase_SH3); or iii) dominant negative CSK comprising a mutation at amino acid position 222 (LT22+dnCSK (K222R)) showed increased production of IL-2 following co-culture with CD22-expressing target cells.

T-cells expressing the CD22 CAR alone produced detectable levels of IFNγ following co-culture with high-expressing CD22 target cells. IFNγ production was completely abolished by the co-expression of wild-type CSK. By contrast, T cells co-expressing any of the dominant negative CSKs, i.e.: i) dominant negative CSK lacking the kinase domain (LT22+dCSK (del_kinase)); ii) dominant negative CSK lacking the kinase domain and SH3 domains (LT22+dCSK (del_kinase_SH3); or iii) dominant negative CSK comprising a mutation at amino acid position 222 (LT22+dnCSK (K222R)), showed increased production of IFNγ following co-culture with CD22-expressing target cells. Production of IFNγ was increased compared to T-cell expressing CD22 CAR alone following co-culture with either high or low-expressing CD22 target cells.

Expression of dominant negative CSK in a CAR-T cell appears to increase the sensitivity of the CAR-T cell, improving cytotoxicity and cytokine release. By contrast, both cytotoxicity and cytokine release are dampened by the over-expression of wild-type CSK in CAR-T cells.

### Example 4—Investigating the Cytotoxicity and Cytokine Release of LT22 CAR Co-Expressed with a Conditionally Inactive CSK Mutant

A panel of CAR-encoding nucleic acid constructs were generated as follows:

LT22_LH-COM P-TyrpTM-41BBz—expressing the CD22 CAR only

LT22_ LH-COM P-TyrpTM-41BBz-2A-wtCSK—co-expressing the CD22 CAR and wild-type CSK

LT22_LH-COMP-TyrpTM-41BBz-2A-dCSK^{AS}—co-expressing the CD22 CAR and conditionally inactive CSK whose catalytic activity is inhibited by the agent 3-iodo-benzyl-PP1.

PBMCs were either left untransduced or transduced with one of the constructs. The capacity of the CAR-T cells to kill target cells expressing CD22 was investigated using a FACS-based killing (FBK) assay. Target cells which did not express CD22 were used as a negative control. Two different types of CD22-expressing SupT1 target cells were generated, one expressing CD22 at a low density (average number of CD22 molecules per cell: 3,309) and one expressing CD22 at a high density (average number of CD22 molecules per cell: 20,262). T-cells were co-cultured with target cells at an effector to target ratio of 1:4 and the data are shown in FIG. 7.

Without the addition of the CSK^{AS} inhibitor, 3-IB-PP1 (0 uM), the inhibition of LT22 CAR T cell cytotoxicity by CSK^{AS} was significant. However, upon 3-IB-PP1 administration (10 uM), the cytotoxicity of T cells expressing CSK^{AS} against SupT1 CD22 High or SupT1 CD22 Low targets was significantly increased compared to the media condition. Furthermore, in the presence of the inhibitor, the CAR bearing CSK^{AS} showed a significant increase in sensitivity compared to LT22 CAR.

In order to investigate cytokine release, supernatant was taken at 72 h post co-culture set up and analysed by ELISA for the presence of IL-2 and IFN-γ.

The results for IL-2 release are shown in FIG. 8. The addition of 3-IB-PP1 (10 uM) led to the CSK^{AS} expressing T cells releasing increased levels of IL-2 against SupT1 CD22 High or SupT1 CD22 Low targets.

The results for IFN-γ release are shown in FIG. 9. In the media condition (0 nM), the IFN-γ release by T Cells was inhibited by both wtCSK and CSK^{AS}. However, upon the addition of 3-IB-PP1 (10 uM), T cells expressing CSK^{AS} and co-cultured with SupT1 CD22 High or SupT1 CD22 Low targets produced significantly higher IFN-γ concentrations compared to the media condition (0 uM). Furthermore, in the presence of the inhibitor, the CAR bearing CSK^{AS} displayed a significant increase in the release of IFN-γ compared to the LT22 CAR. In a similar manner to their cytotoxic capacity, inhibition of CSK^{AS} led to increased cytokine release upon antigen stimulation with high and low densities.

### Example 5—Investigating the Cytotoxicity and Cytokine Release of LT22 CAR Co-Expressed with a Conditionally Inactive CSK Mutant (CSK^{AS}) in Response to Increasing Concentrations of the CSK^{AS} Inhibitor, 3-IB-PP1

The co-culture described in Example 4 was repeated, but this time with increasing concentrations of 3-IB-PP1 (0.156 uM-10 uM). The SupT1 CD22 high targets had an average 6,309 CD22 molecules per cell. The FBK results are shown in FIG. 10. Without the addition of the CSK^{AS} inhibitor, 3-IB-PP1 (0 uM), the inhibition of LT22 CAR T cell cytotoxicity by CSK^{AS} was notable. However, upon the administration of increasing concentrations of 3-IB-PP1 (0.156 uM-10 uM), the cytotoxicity of T cells expressing CSK^{AS} against SupT1 CD22 High targets increased in a dose-dependent manner.

The results for IFN-γ release are shown in FIG. 11. Without the addition of the CSK^{AS} inhibitor (0 uM), the inhibition of IFN-γ release by LT22 CAR T cells co-expressing CSK^{AS} was notable. However, upon the administration of increasing concentrations of 3-IB-PP1 (0.156 uM-10 uM), IFN-γ production by T cells expressing CSK^{AS} against SupT1 CD22 High targets increased in a dose-dependent manner.

### Example 6—Generation of an Inducible CSK Dampener

A panel of CAR-encoding nucleic acid constructs are generated as follows:

| Construct | Expresses |
|---|---|
| LT22_LH-COMP-TyrpTM-41BBz | CD22 CAR only |
| LT22_LH-COMP-TyrpTM-41BBz_ | CD22 CAR and inducible |
| 2A_CSK(SH3-SH2)- | CSK dampener system |
| L-FKBP_2A_CSK(kinase)-L-FRB | G1.3 illustrated in FIG. 12a |
| LT22_LH-COMP-TyrpTM-41BBz_ | CD22 CAR and inducible |
| 2A_CSK(SH3-SH2)- | CSK dampener system |
| L-FKBP_2A_FRB-L-CSK(kinase) | G1.1 illustrated in FIG. 12a |
| LT22_LH-COMP-TyrpTM- | CD22 CAR and inducible |
| 41BBz_2A_FKBP-L- | CSK dampener system |
| CSK(SH3-SH2)_2A_CSK(kinase)-L-FRB | G1.2 illustrated in FIG. 12a |
| LT22_LH-COMP-TyrpTM- | CD22 CAR and inducible |
| 41BBz_2A_FKBP-L- | CSK dampener system |
| CSK(SH3-SH2)_2A_FRB-L-CSK(kinase) | G1.4 illustrated in FIG. 12a |

PBMCs are either left untransduced or transduced with one of the constructs. The capacity of the CAR-T cells to kill target cells expressing CD22 is investigated using a FACS-based killing assay. Target cells which did not express CD22 were used as a negative control. Two different types of CD22-expressing SupT1 target cells were generated, one expressing CD22 at a low density and one expressing CD22 at a high density. T-cells are co-cultured with target cells at effector to target ratios of 1:2 and 1:4 in the presence of various concentrations of rapamycin. Assays are carried out in a 96-well plate in 0.2 ml total volume using 5×10⁴ transduced target cells per well. The co-cultures are set up after being normalised for transduction efficiency. FBK and cytokine release is assayed after 72 h of incubation.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell which co-expresses:
i) a chimeric antigen receptor (CAR); and
(ii) a dominant negative C-terminal Src kinase (dnCSK).

2. A cell according to claim 1, wherein the dnCSK is:
i) a truncated CSK which is recruited to the cell membrane but lacks a functional kinase domain;
ii) a mutated CSK which lacks the capacity to phosphorylate Y505 of Lck; or
iii) a mutated CSK whose catalytic activity is inhibited by an agent.

3. A cell according to claim 2, wherein the dnCSK is a truncated CSK which retains the capacity to bind PAG, Lime and/or Dok1/2 but which lacks a functional kinase domain.

4. A cell according to claim 3, wherein the dnCSK comprises a CSK SH2 domain but lacks a functional kinase domain.

5. A cell according to claim 4, wherein the dnCSK comprises a CSK SH2 domain and a first dimerisation domain, and wherein the cell co-expresses a dampening component comprising a CSK kinase domain and a second dimerization domain which, in the presence of a chemical inducer of dimerization (CID), binds the first dimerisation domain of the dnCSK.

6. A cell according to claim 6, wherein one dimerization domain comprises an FK506-binding protein (FKBP), the other dimerization domain comprises an FRB domain of mTOR and the CID is rapamycin or a rapamycin analogue.

7. A cell according to claim 2, wherein the dnCSK lacks the capacity to phosphorylate Y505 of Lck and has a substitution or deletion at amino acid position K222.

8. A cell according to claim 2, wherein the dnCSK is a mutated CSK whose catalytic activity is inhibited by 3-iodo-benzyl-PP1.

9-11. (canceled)

12. A nucleic acid construct, which comprises:
a first nucleic acid sequence encoding a chimeric antigen receptor; and
a second nucleic acid sequence encoding a dominant negative C-terminal Src kinase (dnCSK).

13. A nucleic acid construct, which comprises:
a first nucleic acid sequence encoding a chimeric antigen receptor; and
a second nucleic acid sequence encoding a dominant negative C-terminal Src kinase (dnCSK) which comprises a CSK SH2 domain and a first dimerization domain; and
a third nucleic acid sequence encoding a dampening component comprising a CSK kinase domain and a second dimerization domain which, in the presence of a chemical inducer of dimerization (CID), binds the first dimerization domain of the dnCSK.

14. A vector which comprises a nucleic acid construct according to claim 12.

15. A vector which comprises a nucleic acid construct according to claim 13.

16. A pharmaceutical composition comprising a plurality of cells according to claim 1.

17. (canceled)

18. A method for treating a disease, which comprises the step of administering a pharmaceutical composition according to claim 16 to a subject.

19. A method according to claim 18, which comprises the following steps:
(i) isolation of a cell containing sample from a subject;
(ii) transduction or transfection of the cells with a nucleic acid construct; and
(iii) administering the cells from (ii) to the subject.

20. (canceled)

21. A method according to claim 18, wherein the disease is cancer.

22. A method for making a cell which co-expresses:
i) a chimeric antigen receptor (CAR); and
(ii) a dominant negative C-terminal Src kinase (dnCSK), which comprises the step of introducing a nucleic acid construct according to claim 12 into the cell ex vivo.

23. A method for making a cell which co-expresses:
i) a chimeric antigen receptor (CAR); and
(ii) a dominant negative C-terminal Src kinase (dnCSK)
which comprises the step of introducing a nucleic acid construct according to claim 13 into the cell ex vivo.

24. A method for enhancing the target-antigen sensitivity of a CAR-expressing cell according to claim 1 in a subject, wherein the cell expresses a mutated CSK whose catalytic activity is inhibited by an agent, which comprises the step of administering the agent to the subject.

25. A method for dampening CAR-mediated activation of a cell according to claim 5 or 6 in a subject, which comprises the step of step of administering the CID to the subject.
